(19) United States
(12) Patent Application Publication
(10) Pub. No.: US 2011/0033379 A1
(43) Pub. Date: Feb. 10, 2011

(54) RADIO-LABELED MATERIALS AND METHODS OF MAKING AND USING THE SAME

(76) Inventors: John V. Frangioni, Wayland, MA (US); Preeti Misra, Chestnut Hills, MA (US)

Correspondence Address: FISH & RICHARDSON P.C. (BO), P.O. BOX 1022, MINNEAPOLIS, MN, 55440-1022, US

(21) Appl. No.: 12/439,254

(22) PCT Filed: Aug. 29, 2007
(86) PCT No.: PCT/US2007/077155

§ 371 Date: Apr. 22, 2010

Related U.S. Application Data
(60) Provisional application No. 60/841,171, filed on Aug. 29, 2006.

(51) Int. Cl. A61K 51/04; C07F 5/00; C07F 13/00

(52) U.S. Cl. 424/1.65; 540/465; 556/1

(57) ABSTRACT

Radio-labeled materials, e.g., compounds and compositions, methods of making the radio-labeled materials, and applications of the same are disclosed. For example, novel solid-state methods are disclosed that produce radio-labeled compositions that include a reactive, but stable radio-labeled compound in a polar anhydrous solvent. The radio-labeled compounds can be readily conjugated with a variety of ligands.

### CROSS-REFERENCE TO RELATED APPLICATION

This application claims priority from U.S. Provisional Application Ser. No. 60/841,171, filed on Aug. 29, 2006, which is incorporated herein by reference in its entirety.

### STATEMENT AS TO FEDERALLY SPONSORED RESEARCH

This invention was made with Government support under NIH Grant No. R21/R33-CA-88245. Thus, the Government has certain rights in the invention.

### TECHNICAL FIELD

This invention relates to radio-labeled materials, and methods of making and using the same.

### BACKGROUND

Prostate cancer is among the most common malignancies for which healthcare intervention is sought worldwide and, in many Western countries, prostate cancer is the most common noncutaneous malignancy (see, e.g., Gronberg et al., Lancet (2003) 361:859-64, and Potosky et al., Epidemiol. Rev (2001) 23: 181-86). Prostate cancer is currently diagnosed by sector biopsy in men with an elevated serum prostate-specific antigen (PSA) level. As with all biopsies, sector biopsy for prostate cancer is invasive and limited by sampling error. As with other cancer surgeries, biopsies are often still performed without any intraoperative image guidance. More accurate staging would facilitate treatment decisions, and potentially lead to a better outcome for patients. For this reason, molecules that target prostate-specific membrane antigen (PSMA) have been the subject of intense research. To date, PSMA-specific antibodies (see, e.g., Liu et al., Cancer Res., (1997)57:3629-34), aptamers (see, e.g., Lupold et al., Cancer Res. (2002) 62:4029-33), peptides, peptide derivatives (see, e.g., Liu et al., Cancer Res. (2002) 62:5470-75), and small molecules (see, e.g., Pomper et. al, Mol. Imaging (2002) 1:96-101) have been described. From these have emerged radio-labeled antibodies for imaging (see, e.g., Smith-Jones et al., Cancer Res. (2000) 60:5237-43), therapy (see, e.g., McDevitt et al., Science (2001) 294:1537-40), drug filled nanoparticles (see, e.g., Farokhzad et al., Cancer Res. (2004) 64:7668-72), thrombosis-inducing molecules, and targeted chemotherapeutics (see, e.g., Henry et al., Cancer Res. (2004) 64:7995-8001). Low molecular weight ligands, and especially small molecules, are often preferred for tumor targeting due to their rapid biodistribution, rapid clearance, improved tumor penetration, and ease of synthesis.

### SUMMARY

Described herein are simple, cartridge-based, solid-phase prelabeling strategies that rapidly convert readily available and relatively inexpensive radio-labeled starting materials, such as ^{99m}Tc-pertechnetate, into stable, reactive labeling intermediates, such as NHS ester intermediates. These reactive intermediates can be used to quickly label essentially any amine-containing small molecule or peptide in a:single step without the need for employing high-performance liquid chromatography (HPLC).

In general, the invention is related to radio-labeled materials, e.g., compounds and compositions, methods of making the radio-labeled materials, and applications of the same. Many of the radio-labeled materials can be converted into other radio-labeled materials, e.g., by conjugating with small molecules or proteins having a specific affinity for certain cancer cells. Such conjugates can be useful in, e.g., in vivo pathology imaging, such as tumor imaging using single photon emission computed tomography (SPECT) imaging. The novel solid-state methods described herein generally enable the production of stable, but reactive radio-labeled materials in substantially anhydrous solutions, e.g., anhydrous solutions employing polar solvents such as dimethyl formamide (DMF) or dimethyl sulfoxide (DMSO). Since the materials can be rapidly produced, e.g., in less than 25 minutes, they can have a high specific activity. In addition, the methods can provide the materials at a high level of purity, e.g., greater than 95 percent purity.

In one aspect, the invention features methods of making radio-labeled materials by combining a chelating material having a conjugatable group or a protected conjugatable group with a radioactive metal-containing material, a reducing agent, a substantially insoluble crosslinked resin, and a solvent to provide a mixture; reacting the mixture under conditions and for a time sufficient to produce a radio-labeled chelate including the conjugatable group or the protected conjugatable group; and separating the radio-labeled chelate from the mixture.

In some embodiments, the combining is performed by mixing the chelating material in a first solvent with the reducing agent in d second solvent, optionally different from the second solvent, to provide a chelating/reducing agent mixture; adding the chelating/reducing agent mixture to the insoluble crosslinked resin suspended in a third solvent, optionally different from either the first or second solvent, to provide a chelating/reducing agent/resin mixture.

For example, the chelating material can have between 2 and about 8 binding sites, each binding site including a nitrogen, oxygen, or a sulfur atom. For example, the chelating material can include MAS₃ (s-acetylmereaptoacetyltriserine) or MAG₃ (s-mercaptoacetyl-triglycine).

In various embodiments, the conjugatable group can be, e.g., a carboxylic acid group or an alkyl ester thereof. The radioactive metal-containing material can be or can include, e.g., a metal-oxide, such as ^{99m}Tc-pertechnetate. The reducing agent can include a metal, e.g., a metal halide, such as SnCl₂ or a hydrated SnCl₂. The substantially insoluble crosslinked resin can include a plurality of exchange moieties. For example, each can include one or more carboxylate groups.

In some embodiments, the solvent is or includes water, and the conditions and time sufficient to produce the radio-labeled chelate include heating the mixture above about 75° C. for about 10 minutes or more.

In some instances, the combining and reacting are performed in a tubular structure having capacity of less than 3 mL. For example, the combining and reacting can be performed in a tubular structure, and the tubular structure can have a first portion configured to receive the mixture, and a second portion, the first and second portions being separated by a porous member configured to prevent the resin from passing from the first portion into the second portion.

For example, the separating can be performed by eluting the mixture in a manner that the resin is excluded, and the resulting mixture is collected substantially free of the resin. For example, the eluting can be performed by spinning a tubular structure having a first portion containing the mixture, and a second portion for collecting the eluted material, the first and second portions being separated by a porous member configured to exclude the resin.

In some embodiments, the methods further include passing the resulting mixture through a second substantially insoluble crosslinked resin, different from the first resin, in a manner that undesired products are substantially separated from the desired radio-labeled chelate.

In some embodiments, the methods further include eluting the radio-labeled chelate from the second resin using an anhydrous solvent, such as anhydrous DMF or DMSO.

In some instances, the methods further include converting the radio-labeled chelate having a first conjugatable group to a second radio-labeled chelate having a second conjugatable group more reactive than the first conjugatable group by reacting the conjugatable group with one or more reagents to produce a mixture that includes the second radio-labeled chelate. For example, the one or more reagents can include N,N,N′N′-tetramethyl-O-(N-succinimidyl)uronium tetrafluoroborate (TSTU), and diisopropylethylamine (DIEA), and the one or more reactive conjugatable group can include an NHS ester.

In some embodiments, the methods further include passing the mixture that includes the second radio-labeled chelate having the more reactive conjugatable group through one or more resins to remove undesired products, such as unreacted starting materials, side-products or salts, and to provide the second radio-labeled chelate having the more reactive conjugatable group in a high purity form in a polar, substantially anhydrous solvent.

In another aspect, the invention features methods of making radio-labeled materials by combining a chelating material having a conjugatable group or a protected conjugatable group with a radioactive metal-containing material, and a solvent to provide a mixture; reacting the mixture under conditions and for a time sufficient to produce a reaction mixture including a radio-labeled chelate having the conjugatable group or the protected conjugatable group; and separating the radio-labeled chelate from the reaction mixture by passing the reaction mixture through one or more substantially insoluble crosslinked resins.

In some embodiments, the chelating material can have between 2 and about 8 binding sites, each binding site including a nitrogen, oxygen, or sulfur atom. For example, the chelating material can include DOTA-Ser (see FIG. 5). The radioactive metal-containing material can include one or more of In, Y, Gd, Eu, a lanthanide or mixtures of these. For example, the radioactive metal-containing material can be or can include ¹¹¹InCl₃.

In another aspect, the invention features radio-labeled compositions that include a radio-labeled chelate having a conjugatable group or a protected conjugatable group, as described herein, dissolved in an anhydrous solvent. In such aspects generally, the purity of the radio-labeled chelate is greater than 92.5 percent.

For example, the conjugatable group can be an NHS ester and/or the purity of the radio-labeled chelate can be 98.0 percent or greater. In some embodiments, the radio-labeled chelate having the conjugatable group or the protected conjugatable group can be or can include MAS₃(MO), MAS₃(MO)—NHS, ¹¹¹In-DOTA-Ser, or ¹¹¹In-DOTA-NHS. M can be any of the metals described herein.

In another aspect, the invention features radio-labeled materials that include a compound of Structure I (shown below).

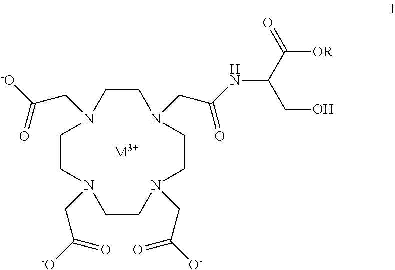

In such aspects, M is In, Y, Gd, Eu, or a lanthanide; and R is; e.g., H, a C1-C10 straight-chain or branched alkyl group, or N-succinimidyl. More generally, RO⁻ is a weaker base than OH⁻, or put another way, RO—H is a stronger acid than water. RO—H has, for example, a pKA or less than 35 when measured in DMSO, e.g., 30, 28, 24, 22, 20, 18, 14, 13, 11, 10, 8, 7 or less, e.g., 5. pKa values for various organic moieties have been tabulated by Bordwell, see, for example, Bordwell et al., Accts. Chem. Research 21, 456 (1988).

In another aspect, the invention features kits for preparing radio-labeled materials that include a chelating material, and one or more cartridges, such as one that includes one or more substantially insoluble crosslinked resin. Such kits can also include one or more reducing agents.

The invention also features systems for making radio-labeled materials that include a chelating material, one or more cartridges, and a reactor for generating a radioactive material that includes a metal.

Aspects and/or embodiments of the invention can have any one of, or combinations of, the following advantages. Generally, the methods used for making the compounds and compositions can provide a practitioner, e.g., a physician or a technician, with on-demand conversion of non-specific radio-labeled intermediates to specific radio-labeled materials, such as membrane specific radio-labeled materials, that is convenient, cost-effective, reproducible, and that reduces the likelihood of human exposure to the radio-labeled compounds. When the compounds and compositions are used as imaging agents, e.g., SPECT imaging agents, they can provide a more specific reagent to certain abnormal cells, e.g., cancer cells, and as a result, can provide better imaging of such abnormal cells. The compounds and compositions can potentially provide earlier detection of the abnormal cells, thus saving lives. More particularly, the methods can provide solutions of radio-labeled materials that arc substantially anhydrous, e.g., dissolved in anhydrous DMSO or DMF, such as DMSO or DMF containing less than about 0.05 percent by weight water, e.g., less than 0.025 percent by weight water, or less than 0.01 percent by weight water. Stable, yet highly reactive compounds can be provided. The methods disclosed are simple and scalable. The methods can be automated. The methods are relatively inexpensive and generally do not require expensive instrumentation.

The methods provide high purity radio-labeled materials. By “purity” we mean radiochemical purity, which is the fraction of the stated isotope present in the stated chemical form, expressed as a percentage. For example, the compounds are produced with a purity greater than 92.5 percent, greater than 95 percent, 97.5 percent, 98 percent, 98.5 percent, 99.0 percent, 99.5 percent, or even greater than 99.9 percent. Radio-labeled materials can be prepared rapidly, e.g., in less than 90 minutes, 70 minutes, 60 minutes, 45 minutes, 30 minutes, 25 minutes, or even less than 20 minutes, providing short half-life materials that have a high specific activity. The methods do not require large amounts of carboxylic acid and/or carboxylic acid salt carriers, such as tartaric acid or tartaric acid salts, that can compete for the desired radio-label. Many of the activated intermediates, such as NHS esters can be conjugated without the need for HPLC purification of the conjugates.

The term “protein” denotes a moiety that comprises a plurality of amino acids, covalently linked by peptide bonds. Proteins can be, e.g., found in nature, or they can be synthetic equivalents of those found in nature, or they can be synthesized, non-natural proteins. In addition to amino acids, a protein can include other moieties, e.g., moieties that include sulfur, phosphorous, iron, zinc and/or copper, along its backbone. Proteins can, e.g., also contain carbohydrate moieties, lipid moieties, and/or nucleic acid moieties. Specific examples of proteins include keratin, elastin, collagen, hemoglobin, ovalbumin, casein, hormones, actin, myosin, annexin V, and antibodies. As used herein, the terms “polypeptide” and “protein” are used interchangeably, unless otherwise stated.

The term “antibody” as used herein) refers to an immunoglobulin molecule or immunologically active portion thereof, i.e., an antigen-binding portion. The antibody can be a polyclonal, monoclonal, recombinant, e.g., a chimeric, de-immunized or humanized, fully human, non-human, e.g., murine, or single chain antibody. In some embodiments the antibody has effector function and can fix complement. In some embodiments, the antibody has reduced or no ability to bind an Fe receptor. For example, the antibody can be an isotype or subtype, fragment or other mutant, which does not support binding to an Fe receptor, e.g., it has a mutagenized or deleted Fe receptor binding region. The antibody can be coupled to a toxin or imaging agent.

“Substantially anhydrous solutions or solvents” are solutions or solvents that generally include less than about 0.1 percent by weight water, e.g., less than 0.05 percent by weight water, less than 0.025 percent by weight water, less than about 0.01 percent by weight water, or even less than about 0.005 percent by weight water.

Unless otherwise defined, all technical and scientific terms used herein have the same meaning as commonly understood by one, of ordinary skill in the art to which this invention belongs. Although methods and materials similar or equivalent to those described herein can be used in the practice or testing of the present invention, suitable methods and materials arc described below. All publications, patent applications, patents, and other references mentioned herein are incorporated by reference herein in their entirety. In case of conflict, the present specification, including definitions, will control. In addition, the materials, methods, and examples arc illustrative only and not intended to be limiting.

Other features and advantages of the invention will be apparent from the following detailed description, and from the claims.

### DESCRIPTION OF DRAWINGS

FIG. 1 is a series of chemical structures of a synthetic path that illustrates a solid phase pre-labeling strategy for the MAS₃ (s-acetylmercaptoacetyltriserine) chelating ligand using an insoluble resin having dicarboxylate functionality; converting the resulting metal chelate [MAS₃(MO)] to a more reactive NHS ester [MAS₃(MO)—NHS]; and then reaction of the more reactive NHS ester with an amino-containing ligand to produce a radio-labeled conjugate [MAS₃(MO)—R].

FIG. 2 is a scheme that illustrates the use of solid phase resins housed in columns and cartridges to label the MAS₃ chelating ligand, and then converting the resulting purified metal chelate [MAS₃(MO)] to a more reactive NHS ester [MAS₃(MO)—NHS] in a polar, substantially anhydrous solvent.

FIG. 3 is a series of chemical structures of GPI (I), Adam-Tri Acid, AdamGPI (II), AdamGPI Dimer (III), and AdamGPI Trimer (IV).

FIG. 4 is a series of chemical structures of a synthetic pathway that illustrates the conjugation of MAS₃(MO)—NHS with GPI (I), AdamGPI (II), AdamGPI Dimer (III) and AdamGPI Trimer (IV), producing compounds (Ia), (IIa), (IIIa), and (IVa), respectively.

FIG. 5 is a series of chemical structures of a synthetic pathway that illustrate a labeling strategy of the DOTA-Ser chelating ligand using, e.g., ¹¹¹InCl₃; purification of the resulting metal chelate (e.g., ¹¹¹In-DOTA) using sad phase resins; converting the metal chelate into a more reactive NHS ester (e.g., ¹¹¹In-DOTA-NHS); and then reaction of the more reactive NHS ester with an amino-containing ligand to, produce a radio-labeled conjugate (e.g., ¹¹¹In-DOTA-R).

FIG. 6 is a scheme that illustrates the use of solid phase resins housed in columns and cartridges to label the DOTA-Ser chelating ligand, and then converting the resulting purified metal chelate, e.g., [¹¹¹In-DOTA-Ser], to a more reactive NHS ester, e.g., [¹¹¹In-DOTA-NHS], in a polar, substantially anhydrous solvent.

FIGS. 7A-7C are C₁₈ HPLC radiochromatographs of, respectively, ^{99m}Tc-MAS₃, its NHS ester, and its NHS ester after incubation in pH 10 buffer for 20 min, while FIG. 7D is an C₁₈ HPLC radiochromatograph of ^{99m}Tc-MAS₃ in the presence of tartrate.

FIGS. 8A and 8B are, respectively, C₁₈ HPLC ELSD tracings (top) and mass spectrographs (bottom) of the identified peak for ¹⁸⁵Re-MAS₃, and its NHS ester (the expected isotopic patterns shown in inset).

FIGS. 9A-9D are HPLC traces for compounds Ia-IVa using either ^{99m}Tc (left) or ¹⁸⁵Re (right). The retention times for compounds Ia-IVa arc shown, as are the ES-TOF mass spectrographs of the peak for ¹⁸⁵Re compounds (insets).

FIG. 10A is an HPLC trace for compound ^{99m}Tc-MAS₃-IVa incubated for 0 (left) or 4 hours (right) at 37° C. in PBS, and FIG. 10B is an HPLC trace for compound ^{99m}Tc-MAS₃-IVa incubated for 0 (left) or 4 hours (right) at 37° C., in 100% serum. Samples in PBS were resolved on a Symmetry C₁₈ column, whereas samples in serum were resolved on a 120-Å pore-size column. FIG. 10C are HPLC tracings showing retention times for the gel-filtration markers M₁-M₅; marker retention times being M₁=6.6 min, M₂=8.2 min, M₃=9.2 min, M₄=11.1 min, and M₅=13.4 min.

FIGS. 11A and 11B are each a series of graphs showing results for a live cell binding assay; in the assay, PSMA-positive LNCaP cells were incubated with monomeric ^{99m}Tc-Ia or trimeric ^{99m}Tc-IVa in the presence of increasing concentrations of the corresponding non-radioactive test compound. Shown are the results for monomeric ¹⁸⁵Re-Ia and trimeric ¹⁸⁵Re-IVa in TBS (left), PBS (middle) and 100% serum (right) (N.D. indicates none detected).

### DETAILED DESCRIPTION

### General Methodology

The novel solid-state methods described herein generally enable the production of stable, but reactive radio-labeled materials in polar, substantially anhydrous solutions. The materials can be rapidly produced, e.g., in less than 25 minutes, and can have a high specific activity, which can maximize signal intensity during in vivo pathology imaging.

In one method of making a radio-labeled material, a chelating material, e.g., MAS₃ (s-acetylmercaptoacetyltriserine), having a conjugatable group or a protected conjugatable group, is combined with a radioactive metal-containing material, such as ^{99m}TcO₄⁻ (pertechnetate), a reducing agent, such as stannous chloride dihydrate, a substantially insoluble crosslinked resin, and a solvent to form a mixture. The mixture is allowed to react under conditions and for a time sufficient to produce a radio-labeled chelate including the conjugatable group or the protected conjugatable group. The radio-labeled chelate is separated from the mixture and any other undesired reaction products, such as starting materials, or salts, forming a solution of the purified radio-labeled chelate. In some instances, the substantially insoluble crosslinked resin has functional groups, such as carboxylate groups, that take the place of the acids and acid salts that are conventionally utilized to make radio-labeled materials. Such resins have the advantage that can be easily removed from the reaction mixture.

The chelating material, radioactive metal-containing material, reducing agent, resin, and solvent can be combined in a number of ways. For example, in some embodiments, the components are combined by mixing the chelating material in a first solvent with the reducing agent in a second solvent, optionally different from the first solvent, to provide a chelating/reducing agent mixture; adding the chelating/reducing agent mixture to the insoluble crosslinked resin, such as a Chelex® resin suspended in a third solvent (optionally different from either the first or second solvent), to provide a chelating/reducing agent/resin mixture.

In some embodiments, the chelating material has between 2 and about 12 binding sites, e.g., between 2 and about 8 binding sites. Each binding site can include, e.g., a nitrogen, oxygen, sulfur, or phosphorus atom. In some embodiments, the chelating material forms a macrocyclic ring, e.g., having between about 10 and about 24 atoms in the ring, e.g., between about 10 and about 18, or between about 12 and 16 atoms in the ring. The chelating material can be monomeric, oligomeric, or polymeric. Examples of monomeric chelating materials include MAS₃ (s-acetylmercaptoacetyltriserine) or MAG₃ (s-mercaptoacetyltriglycine). Examples of oligomeric and polymeric chelating agents include oligomers and polymers that have one or more spaced-apart macrocyclic rings along their backbone.

The conjugatable group can be conjugated, e.g., by a hydroxyl group, an amino group, such as a primary amino group, or a thiol group. In some embodiments, the conjugatable group is a carboxylic acid group, or a alkyl ester thereof, e.g., a methyl, ethyl or isopropyl ester.

In some embodiments, the radioactive metal-containing material is or includes a metal-oxide, such as ^{99m}Tc-pertechnetate, e.g., as its sodium, or potassium salt.

In some instances, the reducing agent is, or includes, a metal, e.g., in the form of a metal halide, such as a metal chloride. For example, the reducing agent can be, or can include, SnCl₂ or a hydrated SnCl₂, such as a dihydrate. Other reducing agents include boron-containing reducing agents, such as sodium borohydride or lithium borohydride.

The solvent can be, or can include water, which can optionally include acids, bases, or buffers dissolved therein. The first, second and third reducing agents can be water, or they can be, e.g., buffered water, e.g., buffered with (N-morpholino)ethanesulfonic acid, acidified water, e.g., acidified with hydrochloric acid.

The resin can be, e.g., crosslinked poly(N-vinyl pyrrolidone), poly(m-divinylbenzene), functionalized crosslinked resins (e.g., functionalized with dicarboxylate groups, such as those available from Bio-Rad), C18 mixed-bed resin, anion exchange resin, or cation exchange resin, such as those available from Waters. These resins can be functionalized so that they can, at least in part, replace acidic, basic oxidizing or reducing solutions, and generally perform cationic and/or anionic exchange with reagents in a solution phase, while they themselves remain undissolved in the solution phase.

Reaction conditions, and time sufficient to produce the radio-labeled chelate can include heating the mixture above about 75° C., e.g., above about 85, or above about 95° C. for about 10 minutes or more, e.g., 15 minutes, or 25 minutes.

In some embodiments, the reaction conditions include utilizing a buffer, such as on that contains 2-(N-morpholino)ethanesulfonic acid (MES). In some embodiments, the pH 5.0 is maintained below about 6.5, such as between 3.0 and about 6.0 or between about 4.0 and 5.0

### Methods Using Cartridges

In some embodiments, the combining and reacting steps are performed in tubular structures, e.g., cartridges having a first portion configured to receive the mixture, and a second portion configured to received the reaction product (radio-labeled compound). A porous member, such as a glass frit, or a polymeric membrane, configured to exclude the resin, can separate the first and second portions. This configuration allows the tubular structure to be spun at a high rate, e.g., at 3,000 rpm or higher, e.g., 5,000 rpm or higher, or even 10,000 rpm or higher, the centrifugal force causing the mixture to pass through the porous material with the exclusion of the resin. Such a configuration is available from BioRad under the Tradename of MICRO-BIOSPIN®. If desired, the combining and reacting can be conveniently performed in a tubular structure or cartridge having a capacity of less than 3 mL, e.g., less than 2 mL, or 1 mL, or even less than 0.8 mL. This small size minimizes the generation of radioactive wastes. Desirably, the tubular structure can be disposable, which can increase safety by minimizing human exposure to radioactive materials.

After collection, the desired radio-labeled compound is free of the resin. If desired, the resulting mixture can be passed through a second substantially insoluble crosslinked resin different from the first resin, such as one bearing different functionality than the first resin (e.g., a cationic resin), in a manner that any undesired products arc substantially separated from the desired radio-labeled chelate. The second substantially insoluble crosslinked resin can be in cartridge form, and if desired, can be disposable. Suitable disposable cartridges are available from BioRad under the tradenames OASIS HLB®, OASIS MAX®, and OASIS MCX®.

In some embodiments, the desired radio-labeled chelate is eluted from the second resin using an anhydrous solvent, e.g., a polar, anhydrous solvent such as DMF and/or DMSO.

In some instances, it can be desirable to convert the radio-labeled chelate having a first conjugatable group to another radio-labeled chelate having a second conjugatable group that is more reactive than the first conjugatable group by reacting the conjugatable group with one or more reagents to produce another mixture including the other radio-labeled chelate and other products. For example, a carboxylic acid group can be converted to a more reactive NHS ester by using, e.g., N,N,N′N′-tetramethyl-O-(N-succinimidyl)uronium tetrafluoroborate (TSTU), and diisopropylethylamine (DIEA).

The radio-labeled chelate having the more reactive second conjugatable group, and other products, such as unreacted materials and/or side products, can be passed through one or more resins to remove undesired products, and to provide the other radio-labeled chelate having the more reactive conjugatable group in a high purity form. If desired, the radio-labeled chelate having the more reactive conjugatable group can be collected in a substantially anhydrous solvent, such as a polar solvent, e.g., DMF or DMSO. Having the radio-labeled compound in an anhydrous condition, and in a polar solvent can be advantageous because it can be easily and rapidly conjugated in this state. Often, the conjugates formed require no purification prior to use.

In some embodiments, the entire process from combining the chelating material, radioactive metal-containing material, reducing agent, resin, and the solvent to collecting the radio-labeled chelate having the more reactive conjugatable group in a substantially anhydrous solvent, can be take less than 60 minutes, e.g., less than 55 minutes, less than 45 minutes, less than 40 minutes, less than 35 minutes, less than 30 minutes, or even less than 25 minutes.

### Radio-Labeled Chelates and Ligands

Any of the radio-labeled chelates described herein having a conjugatable group can be reacted with many different ligands, e.g., targeting ligands. For example, the ligand can have a nucleophilic group, such as a primary amine group, a thiol group, or a hydroxyl group. The ligand can be, e.g., a protein, a protein fragment, a peptide, e.g., octreotide (sandostatin), a low molecular weight peptide, an antibody, a carbohydrate, or an antigen. Possible proteins, protein fragments, low molecular weight peptides, antibodies, carbohydrates, or antigens can be found in G. Hermanson, Bioconjugate Techniques: Academic Press (November 1995, ISBN 012342335X). Additional ligands are discussed in “RADIO-LABELED COMPOUNDS, COMPOSITIONS, AND METHODS OF MAKING THE SAME,” U.S. Ser. No. 11/156,259, filed on Jun. 17, 2005, now Published U.S. Patent Application No. 2006/0083678. Still additional ligands are discussed in “SUBSTITUTED ADAMANTANES, AND METHODS OF MAKING THE SAME”, U.S. Ser. No. 11/222,951, filed on Sep. 9, 2005, now Published U.S. Application No. 2006/0063834. Small molecule targeting ligands are discussed in “MODIFIED PSMA LIGANDS AND USES RELATED THERETO,” U.S. Pat. No. 6,875,886. Any and all of the ligands described in all of these applications and patents can be utilized. The entire contents of each patent and application are incorporated herein by reference in their entirety.

In particular embodiments, TM601, a 36 amino-acid chlorotoxin peptide, which binds selectively to malignant tumors, such as tumors of the brain, breast, prostate, and lung, can be used as the ligand. For example, the NHS ester of ^{99m}Tc-MAS₃ can be conjugated with TM601, to produce, ^{99m}Tc-MAS₃-TM601 conjugate.

### Solid Phase Pre-Labeling Strategies

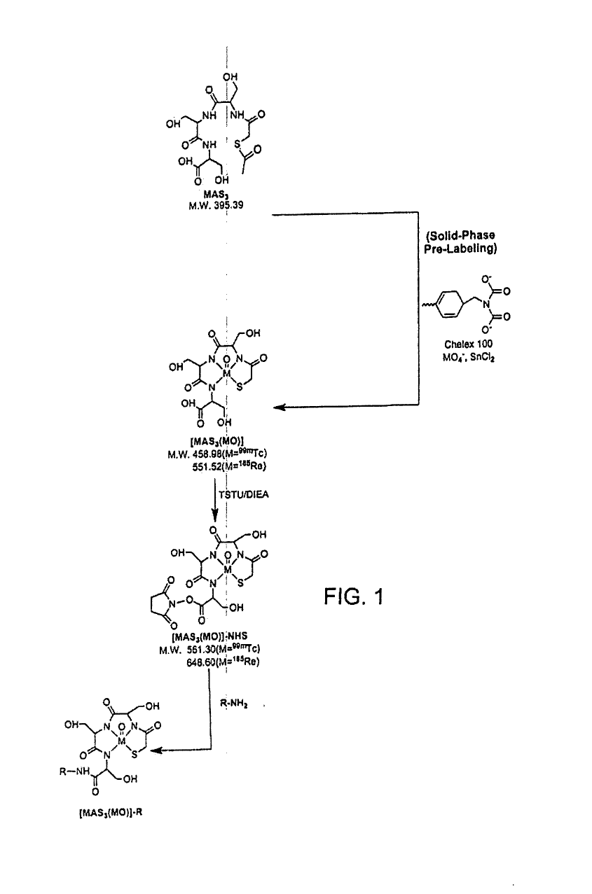

FIG. 1 shows a solid phase pre-labeling strategy for the MAS₃ (s-acetylmercaptoacetyltriserine) chelating ligand using an insoluble resin having dicarboxylate functionality. Briefly, the MAS₃ chelating ligand is combined with a metal oxide, e.g., radioactive ^{99m}Tc-Pertechnetate, or the cold rhenium analog (as surrogate for the radioactive material), a reducing agent such as stannous chloride and Chelex 100 resin. After separation of the resin, the resulting metal chelate [MAS₃(MO)] is collected, and then converted into the more reactive NHS ester [MAS₃(MO)—NHS). The more reactive NHS ester can be reacted with an amino-containing ligand to produce a radio-labeled conjugate [MAS₃(MO)—R].

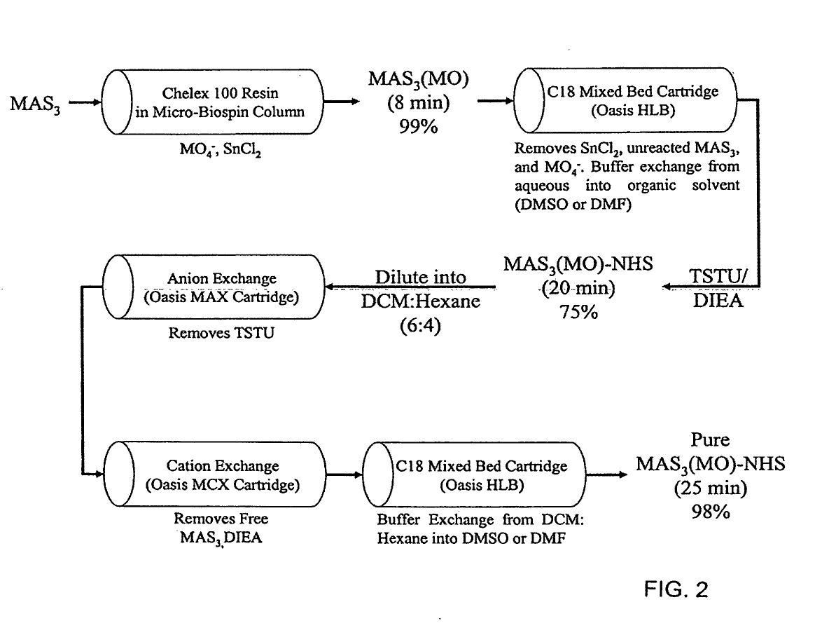

FIG. 2 provides a more detailed view of the conversion of MAS₃ to [MAS₃(MO)—NHS]. In some embodiments, a slurry of CHELEX® 100 resin (Bio-Rad, Hercules, Calif.) in a buffer, e.g., a pH 5.0 buffer, is added to an empty micro BIO-SPIN® (Bio-Rad) chromatography column/tube. MAS₃ is dissolved in water and stannous (II) chloride dihydrate is dissolved in dilute HCl. The MAS₃ is mixed with the tin solution, and then the mixture is added to the CHELEX® resin. When it is desired to label with ^{99m}Tc, ^{99m}Tc-pertechnetate, can be eluted directly from a ⁹⁹Mo generator with saline into the tube, and then the resulting mixture can be allowed to react. The metal chelate solution, e.g., ^{99m}Tc-MAS₃ solution can then be diluted, and passed through an activated OASIS® cartridge (Waters). After washing, any remaining water residues can be purged with nitrogen. The metal chelate, e.g., ^{99m}Tc-MAS₃ can be eluted by washing the cartridge with dry DMF and/or DMSO. The purified metal chelate in DMF and/or DMSO can then be reacted with TSTU and DIEA, producing the NHS ester. The NHS ester can be purified using OASIS MCX® & MAX® cartridges. For purification, the ester can be diluted in dichloromethane:hexane (e.g., 6 weight parts dichloromethane to 4 weight parts hexane) and then it is loaded on Oasis MCX & MAX cartridges attached in series. The purified NHS ester can be collected, and then the solvent can be exchanged from dichloromethane:hexane to DMF and/or DMSO.

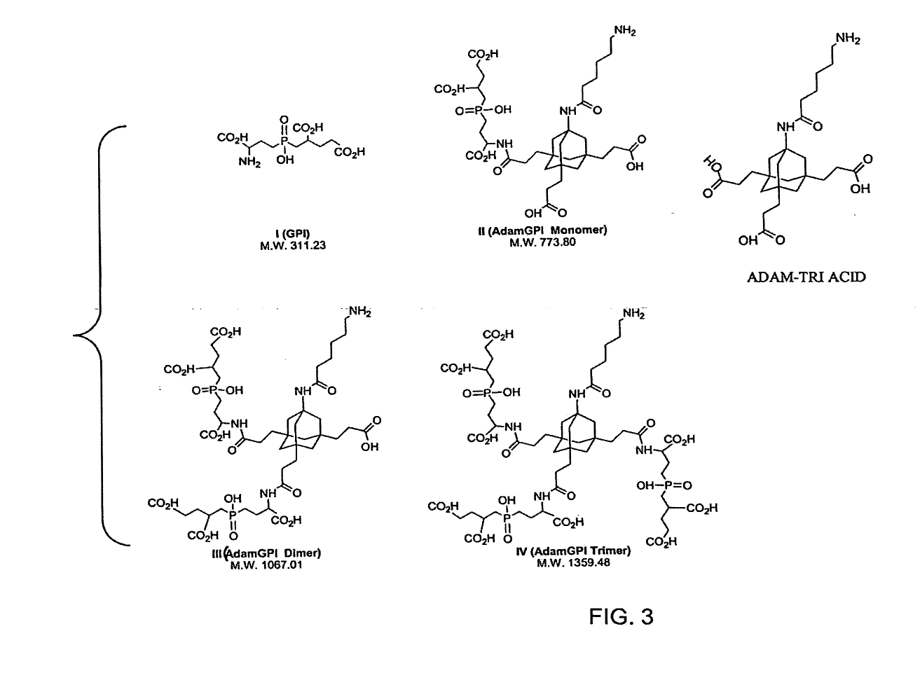

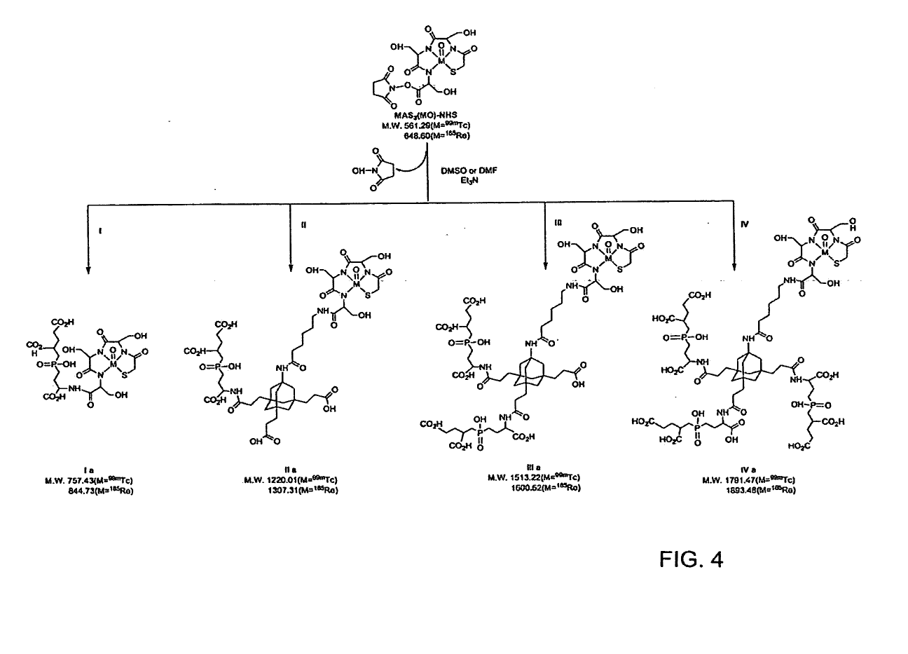

FIG. 3 shows the reaction of targeting ligand GPI (I) with Adam-Tri Acid to produce AdamGPI (II), AdamGPI Dimer (III), and AdamGPI Trimer (IV), which, if desired, can be separated by preparative HPLC. Referring to FIG. 4, conjugation of GPI (I), AdamGPI (II), AdamGPI Dimer (III) and AdamGPI Trimer (IV) with [MAS₃(MO)—NHS] in dry DMSO and/or DMF in the presence of triethylamine, produces, repectively, compounds Ia, IIa, IIIa and IVa, as shown.

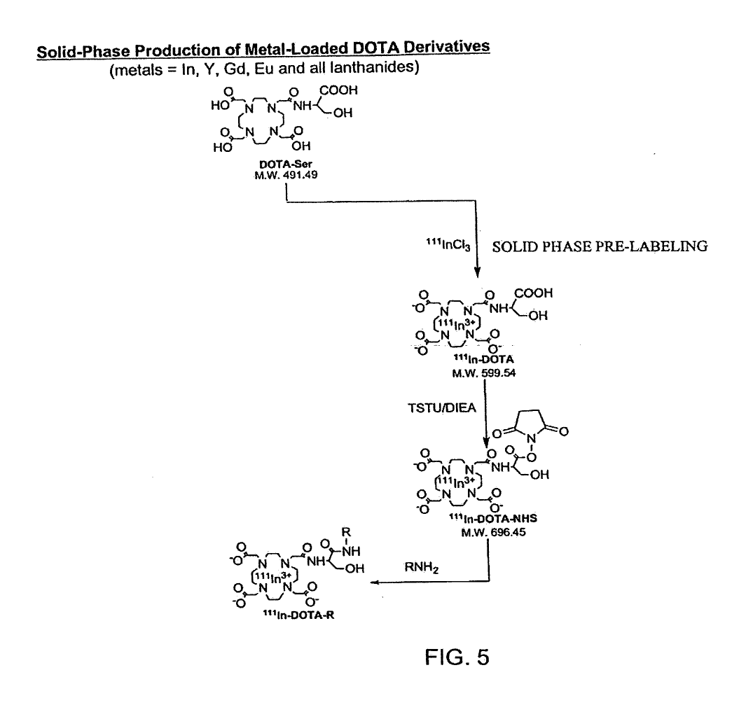

Another method of making radio-labeled materials includes combining a chelating material, such as DOTA-Ser (see, FIG. 5), having a conjugatable group or a protected conjugatable group, with a radioactive metal-containing material, such as ¹¹¹InCl₃, and a solvent to provide a mixture. The mixture is reacted under conditions and for a time sufficient to produce a reaction mixture that includes a radio-labeled chelate having the conjugatable group or the protected conjugatable group. The radio-labeled chelate is separated from the reaction mixture by passing the reaction mixture through one or more substantially insoluble crosslinked resins.

The chelating material can be monomeric, oligomeric, or polymeric. Examples of monomeric chelating materials include DOTA-Ser, MAS₃, or MAG₃. The chelating material can have between 2 and about 12 binding sites, each binding site can, e.g., include a nitrogen, oxygen, sulfur, or phosphorus atom. Some chelating materials are in the form of macrocyclic rings.

The conjugatable group can also be any of the groups described herein.

In some embodiments, the radioactive metal-containing material is, or includes In, Y, Gd, Eu, a lanthanide, or mixtures of these metals. For example, the radioactive metal-containing material can be, or can include ¹¹¹InCl₃.

The mixture can include a buffer, e.g., ammonium acetate, or another material that aids in the reaction, or that aids in driving the reaction to completion.

The solvent can be water, or any other solvent described herein.

In some instances, the separating of the radio-labeled chelate from the reaction mixture includes passing the reaction mixture through a first resin configured to remove any undesired metallic materials, and then passing through a second resin different from the first resin configured to remove other undesired products. Suitable resins and resin configurations are any of those described above. The method can further include eluting the radio-labeled chelate from the second resin using an anhydrous solvent, such as those described herein.

More reactive intermediates can be produced, if desired, by converting the radio-labeled chelate having a first conjugatable group to another radio-labeled chelate having a second conjugatable group more reactive than the first conjugatable group by reacting the conjugatable group with one or more reagents to produce another mixture including the other radio-labeled chelate and other undesired products. Such other mixtures can be purified by passing the mixture including the other radio-labeled chelate through one or more resins to remove undesired products. In some instances, a radio-labeled chelate having the more reactive conjugatable group is provided in a high purity form in a substantially anhydrous solvent.

FIG. 5 shows a solid phase pre-labeling strategy for the DOTA-Ser chelating ligand using insoluble resins. Briefly, the DOTA-Ser chelating ligand is combined with a radioactive metal-containing material, such as ¹¹¹InCl₃, and a solvent to provide a mixture. The mixture is allowed to react, and then the metal chelate (e.g., ¹¹¹In-DOTA) is separated from the reaction mixture on a resin bed to produce the purified metal chelate. The metal chelate can then be converted to a more reactive metal chelate such as ¹¹¹InDOTA-NHS. The more reactive metal chelate can then be converted to a conjugate, such as ¹¹¹InDOTA-R by reaction with an amino-containing material.

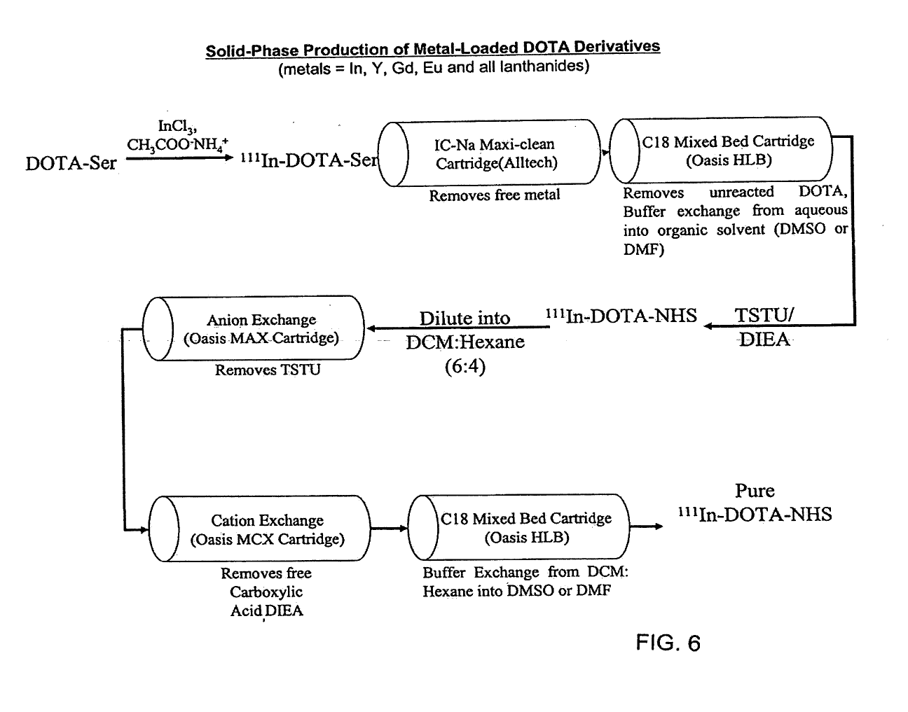

FIG. 6 provides further detail on the conversion of DOTA-Ser to, e.g., [¹¹¹In-DOTA-NHS]. In some embodiments, ¹¹¹InCl₃, ammonium acetate and DOTA-Ser are combined, and allowed to react. The reaction mixture is passed through a first column configured to remove uncombined metal, and then through a second column configured to remove other impurities. The purified chelate can be eluted from the second column using a polar solvent, such as DMSO and/or DMF, so that the purified chelate is dissolved in the polar solvent. The purified metal chelate can then be reacted with TSTU and DIEA, producing the NHS ester. The NHS ester can be purified using OASIS MCX° & MAX® cartridges. For purification, the ester can be diluted in dichloromethane:hexane, and then it was loaded on OASIS MAX® and MCX® cartridges attached in series. The purified NHS ester can be collected, and then the solvent can be exchanged from dichloromethane:hexane to DMF and/or DMSO.

The methods described herein provide radio-labeled compositions that include radio-labeled chelates having a conjugatable group, or a protected conjugatable group, dissolved in an anhydrous solvent, e.g., a polar, anhydrous solvent such as DMSO and/or DMF. The purity of the radio-labeled chelate can be, e.g., greater than 92.5 percent, e.g., greater than 95 percent, 97.5 percent, 98.0 percent, 98.5 percent 99.0 percent, or even greater than 99.5 percent.

The methods are scalable and can be automated to make large quantities of material. For example, a system for making radio-labeled materials on a large scale can include a chelating material, one or more cartridges, e.g., filled with a substantially crosslinked resin, and a reactor for generating a radioactive material that includes a metal. If desired, the resin can be functionalized, e.g., with carboxylate groups, to at least assist in the making of the radio-labeled materials. The system can also include a robot communicating with a computer for combining the chelating material and any other reactants with the radioactive material in specified proportions. For protection, the system can be housed in a protective containment vessel for containment of radiation, and for protection of workers.

### Applications

The radio-labeled compounds can be used to form conjugates that have a specific affinity for certain abnormal cells, e.g., cancer cells, and can be useful, e.g., in in-vivo pathology imaging, e.g., tumor imaging using SPECT. When properly configured, e.g., when the conjugate includes a molecular architecture that can bind specifically to a moiety of interest, the radio-labeled conjugates can be used to specifically image abnormalities of the prostate, bladder, brain, kidneys, lungs, skin, pancreas, intestines, uterus, adrenal gland, and eyes. Antibodies are known that bind specifically to each of these types of tumors, and can be linked to the new materials described herein.

The conjugates can also be used to deliver therapeutic radiation doses to specific locations in the body. For example, the conjugate can include a peptide residue, such as a chlorotoxin peptide residue, which binds selectively to malignant tumors, such as tumors of the brain, breast, prostate, or lung. Such a conjugate can selectively deliver radiation to those tumors to kill and/or reduce their size.

### Examples

Reagents

Guilford (now MGI Pharma, Baltimore, Md.) compound 11245-36 (GPI), 2[((3-amino-3-carboxypropyl)(hydroxy)(phosphinyl)-methyl]pentane-1,5-dioic acid, was synthesized as described previously (see, Guhlke et al., Nuclear Medicine & Biology, 25:621-631 (1998). Ultradry DMSO was purchased from Acros Organics (Geel, Belgium). HPLC grade triethylammnonium acetate, pH 7 (TEAA) was from Glen Research (Sterling, Va.). HPLC grade water was from American Bioanalytic (Natick, Mass.). Triserine was from Bachem (King of Prussia, PA). N-succinimidyl S-acetylthioglycolate (SATA) was from Pierce (Rockford, Ill.). All other chemicals, including N,N,N′N′-tetramethyl-O-(N-succinimidyl)uronium tetrafluoroborate (TSTU), and diisopropylethylamine (DIEA) were purchased from Fisher Scientific (Hanover Park, Ill.) and were AGS or HPLC grade.

HPLC/Mass Spectrometry Platform

The HPLC/mass spectrometry platform used for purification of both non-radioactive and radioactive tumor-targeting small molecules and peptides has been described in detail previously (see, e.g., Humblet et al., Mol. Imaging, 4(4):448-462, 2005). Briefly, the system is composed of a Waters (Milford, Mass.) model 1525 binary pump, model 2487 UV detector (Waters), SEDEX® model 75 (Richards Scientific, Novato, Calif.) evaporative light scatter detector (ELSD) with the nebulizer modified to reduce band broadening at low flow rates, a model FC-3200 high-sensitivity PMT gamma detector (Bioscan, Washington, D.C.), and a Waters fraction collector, all housed within a CAPINTEC® (Ramsey, N.J.) hot cell equipped with a model CRC-15R (CAPINTEC®) dose calibrator. For non-radioactive reactions, column eluent was split into a Waters LCT electrospray time-of-flight (ES-TOF) mass spectrometer.

Synthesis of S-Acetylmercaptoacetyltriserine (MAS₃)

14 mg (36 μmol) triserine was dissolved in 350 μL of water. 1 equivalent (3.6 mg, 5 μL) of the base triethylamine (Et₃N) was added, followed by 2 equivalents (16 mg, 72 μmol dissolved in 160 μL DMF) of SATA. The reaction mixture was vortexed at room temperature for 3 h. An additional equivalent (8 mg, 36 μmol dissolved in 80 μL DMF) of SATA was then added and vortexing was continued for an additional 2 h.

To confirm completion of the reaction, a 10 μL sample was analyzed by reverse phase HPLC using a 4.6×150 mm SYMMETRY® (Waters) C₁₈ column and a gradient consisting of 0% to 15% B over 35 min at 1 ml/min, where A=H₂O+0.1% trifluoroacetic acid (TFA) and B=acetonitrile+0.1% TFA. MAS₃ elutes at Rₜ=11.60 min as detected by the ELSD, with its mass confirmed by ES-TOF mass spectrometry. Preparative purification was performed on an HPLC system described in detail previously (see, e.g., Humblet et al., Mol. Imaging, 4(4):448-462, 2005) after dilution into a final volume of 5 ml of H₂O+0.1% TFA. The column was a 19×150 mm SYMMETRY® (Waters) C₁₈ column equipped with a 5 ml sample loop. The gradient consisted of 0% B for 3.5 min, then 0% to 15% B over 35 min at 7 ml/min, where A=H₂O+0.1% TFA and B=acetonitrile+0.1% TFA. MAS₃ eluted at Rₜ=21.80 min using ELSD detection. Fractions containing product were pooled and lyophilized. MAS₃ was obtained as a white powder in 57% isolated yield (8.0 mg, 20.5 μmol), with expected mass confirmed by ES-TOF mass spectrometry, and purity ≧98%.

Solid-Phase Labeling of MAS₃ with ^{99m}Tc-Pertechnetate

50 μL of a 50% slurry of Chelex 100 resin (Bio-Rad, Hercules, Calif.) in 50 mM 2-(N-morpholino)ethanesulfonic acid (MES) buffer, pH 5.0 was added to an empty micro BIO-SPIN® (Bio-Rad) chromatography column/tube, washed once with MES buffer, and centrifuged at 3,000 rpm for 10 seconds. 1.2 mg (8.3 mmol) of MAS₃ was dissolved in 1 ml of water (solution A). 4 mg (1.7 mmol) of stannous (II) chloride dihydrate was dissolved in 1 ml 10 mM HCl (solution B). 100 μL of solution A (830 μmol) and 35 μL of solution B (60 μmol) were mixed well and added to the CHELEX® resin. 5-10 mCi of ^{99m}Tc-pertechnetate, eluted directly from a ⁹⁹Mo generator with saline, was added to the tube. The tube was capped and heated for 10 min in a boiling water bath. ^{99m}Tc loading of MAS₃ was monitored by reverse phase HPLC using a 4.6×150 min SYMMETRY® (Waters) C₁₈ column with a gradient of 0 to 60% B over 30 min at 1 ml/min, where A=10 mM TEAA and B=absolute MeOH. ^{99m}Tc-MAS₃ eluted at Rₜ=14.1 min.

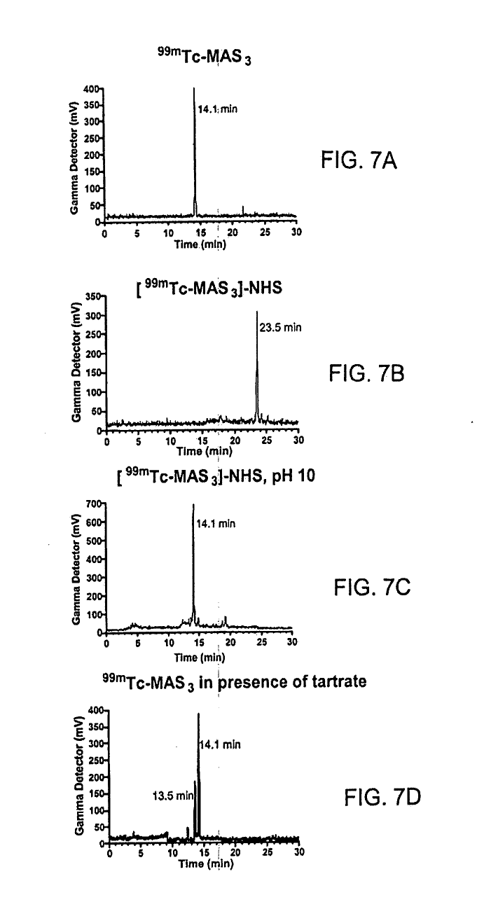

The ^{99m}Tc-MAS₃ solution was diluted with 1 mL water pH=4.0, passed through an activated OASIS® cartridge (Waters), and the column was washed with 10 mL acidified water. Remaining water residues were purged with nitrogen, and ^{99m}Tc-MAS₃ eluted with 0.4 mL dry DMF/DMSO. The confirmation of the compound was analyzed using RP-HPLC. RP-HPLC showed the compound to have a retention time of 14.1 min (see FIG. 7A).

Synthesis of NHS ester of ^{99m}Tc-MAS₃

Purified ^{99m}Tc-MAS₃ in DMF in a reaction vial, 2 equivalents of TSTU and 3 equivalents of DIEA were added. The resulting reaction mixture was stirred at 60° C. for 10 minutes. After the ester formation was complete, it is purified using OASIS MCX® & MAX® cartridges. For purification, the ester was diluted in 6:4 dichloromethane and hexane, and then it was loaded on OASIS MCX® & MAX® cartridges attached in series. The purified NHS ester was collected, and the solvent exchanged from dichloromethane:hexane to DMF or DMSO. Further confirmation was performed by running the ester on a C₁₈ column. RP-HPLC showed the compound to have a retention time of 23.5 min (see FIG. 7B). Hydrolysis of ^{99m}Tc-MAS₃ in solution at pH 10 regenerated ^{99m}Tc-MAS₃, as indicated by the single peak centered about a retention time of about 14.1 min (FIG. 7C). The addition of tartrate to a solution of purified ^{99m}Tc-MAS₃ showed the presence of an additional species, as indicated by peak centered at a retention time of about 13.5 min, indicating that tartrate can effectively compete for radio-label (see FIG. 7D).

Synthesis of ¹⁸⁵Re-MAS₃

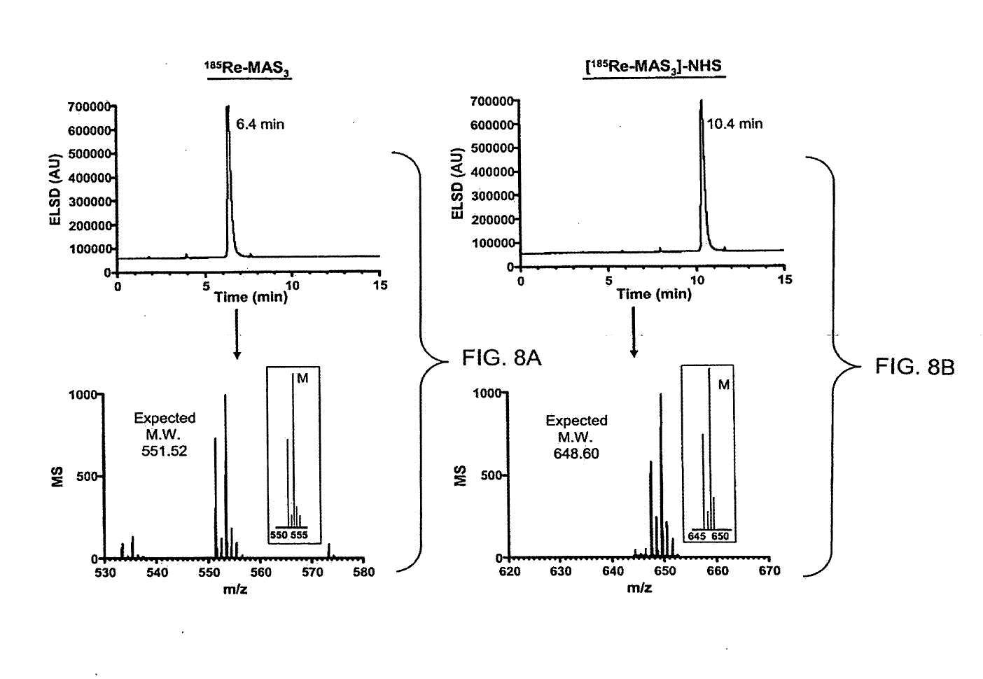

A procedure reported previously (see, Chang et al., Applied Radiation and Isotopes, 50:723-732, 1999) was employed with modification. The MAS₃ (4.8 mg, 8.3 mmol) was dissolved in 1.5 ml of water. Stannous chloride (9 mg, 26.5 mmol) in 1.5 ml of 0.1M citrate buffer (pH 5.0) and “NaReO₄⁻ (6.6 mg, 17 mmol) in 1.5 ml water were added to the MAS₃ solution. The reaction mixture was stirred at 90° C. for 1 h. After the reaction mixture cooled to room temperature, Re-MAS₃ was purified by passing it on OASIS HLB® cartridge, and eluted with DMSO; the fraction containing compound ¹⁸⁵Re-MAS₃ was confirmed by running the LC/MS by monitoring the ELSD and mass spectrometry. ESI-MS calculated for C₁₁H₁₅N₃O₉ReS (M+H)⁺: m/z 553. Found 553. FIG. 8A shows a C₁₈ HPLC ELSD tracing (top) and mass spectrograph (bottom) of the identified peak for ¹⁸⁵Re-MAS₃, centered at 6.4 min (the expected isotopic patterns shown in inset).

Synthesis of [¹⁸⁵Re-MAS₃]-NHS

To synthesize the NHS ester, 0.05 ml of 60 mM TSTU in DMSO was added 0.2 ml of 10 mM Re-MAS₃, followed by 0.025 ml of 200 mM diisopropylethyl amine. The reaction mixture was vortexed at room temperature for 40 min. After completion of the reaction, the mixture was diluted with dichloromethane and purified using OASES MCX®/MAX®/HLB® cartridges. Final confirmation of the NHS ester was performed with LC/MS by monitoring ELSD and mass spectrometry. ESI-MS calculated for C₁₅H₁₈N₄O₁₁ReS (M): m/z 649. Found 649. FIG. 8B shows a C₁₈ HPLC ELSD tracing (top) and mass spectrograph (bottom) of the identified peak for [¹⁸⁵Re-MAS₃]-NHS centered at 10.4 min (the expected isotopic patterns shown in inset).

Synthesis and Purification of [¹⁸⁵Re-MAS₃]-conjugates

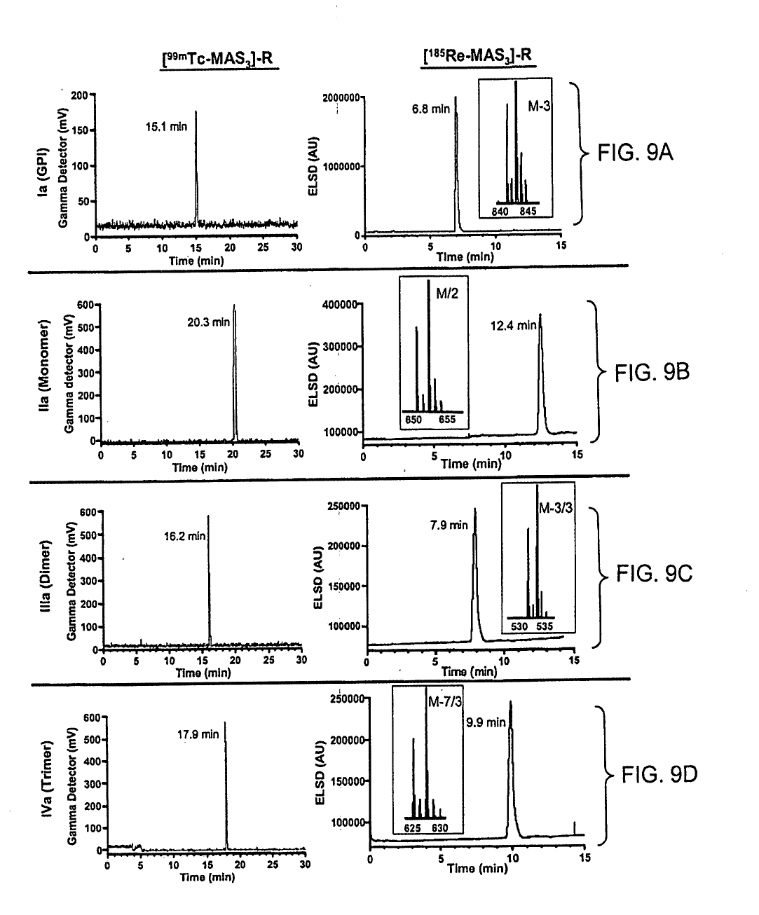

Covalent conjugation of GPI derivatives with [¹⁸⁵Re-MAS₃]-NHS was performed by the addition of 0.1 ml of 100 mM triethylamine in dry DMSO to 0.1 ml of a 10 mM solution of GPI derivatives in dry DMF/DMSO followed by addition of 0.2 ml 10 mM NHS ester of ¹⁸⁵Re-MAS₃ in dry DMSO by constant stirring at room temperature for 2 h. The conjugated ligands were analyzed by LC/MS equipped with Symmetry C₁₈ column (4.5×75, 3 um particle size) and a ELSD. Solvent A was water +0.1% formic acid and solvent B was absolute acetonitrile +0.1% formic acid with linear gradient from 0% to 50% solvent B in 15 min, beginning at 2 min after injection with a flow rate of 1 ml/min. GPI-monomer eluted with a retention of time 6.8 min, while the Adamantane GPI-trimer eluted with a retention time of 9.9 min from the start of the gradient. FIGS. 9A-9D (right) are HPLC traces for conjugates Ia-IVa. The retention times for compounds Ia-IVa are shown, as are the ES-TOF mass spectrographs (insets).

Synthesis and Purification of Radiolabeled PSMA Ligands

Covalent conjugation of GPI derivatives with ^{99m}c-MAS₃-NHS was performed by the addition of 0.1 ml of 100 mM triethylamine in dry DMSO to 0.1 ml of a 10 mM solution of GPI derivatives in dry DMF/DMSO followed by addition of 0.2 ml NHS ester of ^{99m}Tc-MAS₃ (5-7 mCi) in dry DMSO by constant stirring at room temperature for 1 to 2 h. The radiolabeled ligands were purified by reverse phase HPLC chromatography system equipped with a SYMMETRY® C₁₈ column (4.5×75, 3 um particle size) and a radioactivity detector. Solvent A was 10 mM TEAA and solvent B was absolute methanol with linear gradient from 0% to 60% solvent B in 25 min, beginning at 2 min after injection with a flow rate of 1 ml/min. GPI-monomer eluted with a retention of time 15.1 min, and GPI-Adam trimer eluted with a retention time of 17.9 min from the start of the gradient. The materials were used without further concentrating. FIGS. 9A-9D (left) are HPLC traces for conjugates Ia-IVa. The retention times for compounds Ia-IVa are shown.

Quantification of Serum Stability

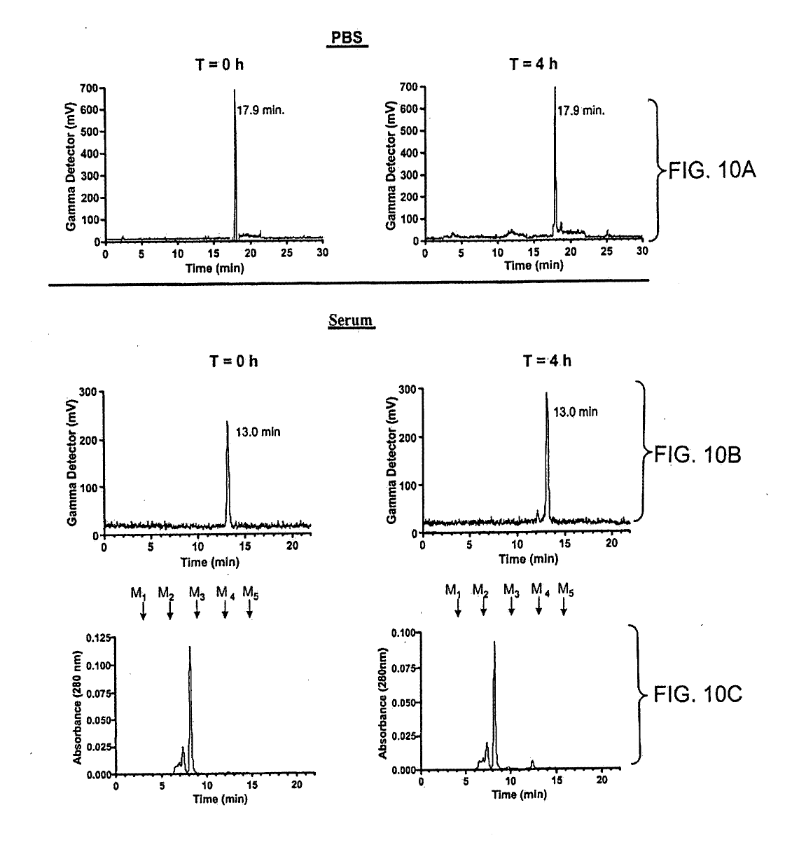

Referring to FIG. 10A and 10B, stability of ^{99m}Tc-MAS₃-GPI compounds was tested by incubation in the absence (PBS only, FIG. 10A) or presence of 100% calf serum (FIG. 10B) for 4 hours at 37° C. Stability and transmetallation were quantified using high-resolution gel-filtration chromatography. For experiments in PBS, a SYMMETRY® C₁₈ column was used, for experiments in serum, whereas samples in serum were resolved on a 120-Å pore-size column with mass separation range of 10 kDa to 1100 kDa. Referring to FIG. 10C, columns were calibrated using a mixture of molecular weight markers: M₁=thyroglobulin (670 kDa), M₂=γ-globulin (158 kDa), M₃=ovalbumin (44 kDa), M₄=myoglobin (17 kDa), and M₅=vitamin B₁₂ (1.3 kDa).

High-Throughput, Radioactive Live Cell Binding and Affinity Assay

Human prostate cancer cell lines LNCaP and PC-3 were obtained from the ATCC (Manassas, Va.). Human bladder cancer cell line TsuPR1 was cultured at 37° C. under humidified 5% CO₂ in RPMI 1640 medium (Mediatech. Cellgro, Herndon, Va.) supplemented with 10% fetal bovine serum (Gemini Bio-Products, Woodland, Calif.) and 5% penicillin/streptomycin (Cambrex Bioscience, Walkersville, Md.). Cells were split onto 96-well filter plates (model MSHAS4510, Millipore, Bedford, Mass.) and grown to 50% confluence (approximately 35,000 cells per well) over 48 hours.

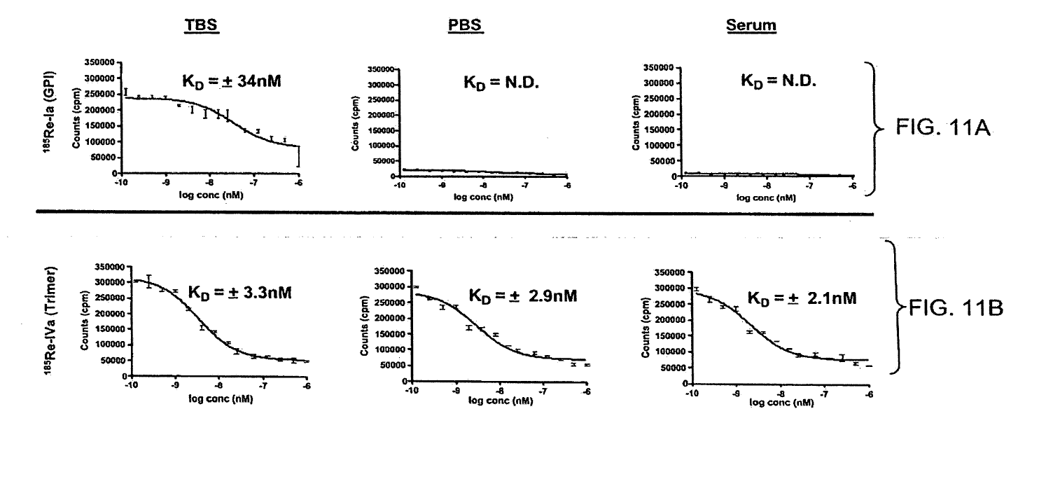

To assign absolute affinity to each compound, a competitive displacement assay was employed using either compound Ia (^{99m}Tc-MAS₃-GPI monomer), or compound IVa (^{99m}Tc-MAS₃-Adamantane GPI trimer), along with non-radioactive test compound. To avoid internalization of the radioligand due to constitutive endocytosis, {Humblet, 2005} live cell binding was performed at 4° C. Referring to FIGS. 11A and 11B, cells were washed 2 times with ice-cold tris-buffered saline (TBS), pH 7.4 and incubated for 20 min at 4° C. with 0.5 μCi of radiotracer in the presence or absence of the test compound. Cells were then washed 3 times with TBS and the well contents transferred directly to 12×75 mm plastic tubes placed in gamma counter racks. Transfer was accomplished using a modified (Microvideo Instruments, Avon, Mass.) 96-well puncher (Millipore MAMP09608) and disposable punch tips (Millipore MADP19650). Well contents were counted on a model 1470 WALLAC WIZARD® (Perkin Elmer, Wellesley, Mass.) ten-detector gamma counter.

^{99m}Tc-MAS₃-TM601 Conjugate

The NHS ester of ^{99m}Tc-MAS₃ was prepared as described above. TM601 was radiolabeled using the NHS ester of ^{99m}Tc-MAS₃, in one step, in DMSO supplemented with a molar excess of triethylamine, and then the product was purified to homogeneity by HPLC. Affinity and Bmax for various cancer cell lines were measured using the described high-throughput live cell binding assay. The specific activity of ^{99m}Tc-MAS₃-TM601 was 3,133 Ci/mmol. In human tumor lines U-87 MG, PC-3, and A549, ^{99m}Tc-MAS₃-TM601 had an affinity ranging from 10-18 nM and a Bmax ranging from 14,000 to 19,000 binding sites per cell. No binding was detected in non-transformed human fibroblasts, mouse NIH 3T3 fibroblasts, or NIH-3T3 cells transformed with H-ras. After intravenous injection, ^{99m}Tc-MAS₃-TM601 cleared rapidly from the blood, with a beta phase half-life of 30.3 min. At 4 hr, 27% of the injected dose was excreted into urine, and 3.6% ID remained in liver.

### Other Embodiments

A number of embodiments have been described. Nevertheless, it will be understood that various modifications may be made without departing from the spirit and scope of the invention. Other embodiments are within the scope of the following claims.

1. A method of making a radio-labeled material, the method comprising:
combining a chelating material having a conjugatable group or a protected conjugatable group with a radioactive metal-containing material, a reducing agent, a substantially insoluble crosslinked resin, and a solvent to provide a mixture;
reacting the mixture under conditions and for a time sufficient to produce a radio-labeled chelate having the conjugatable group or the protected conjugatable group; and
separating the radio-labeled chelate from the mixture.

2. The method of claim 1, wherein the combining is performed by mixing the chelating material in a first solvent with the reducing agent in a second solvent, optionally different from the first solvent, to provide a chelating/reducing agent mixture; adding the chelating/reducing agent mixture to the insoluble crosslinked resin suspended in a third solvent, optionally different from either the first or second solvent or both, to provide a chelating/reducing agent/resin mixture.

3. The method of claim 1, wherein the chelating material has from 2 to about 8 binding sites, each comprising a nitrogen, oxygen, or a sulfur atom.

4. The method of claim 1, wherein the chelating material comprises MAS_{S} (s-acetylmercaptoacetyltriserine) or MAG₃ (s-mercaptoacetyltriglycine).

5. The method of claim 1, wherein the conjugatable group is a carboxylic acid group or an alkyl ester thereof.

6. The method of claim 1, wherein the radioactive metal-containing material comprises a metal-oxide.

7. The method of claim 1, wherein the radioactive metal-containing material comprises ^{99m}Tc-pertechnetate.

8. The method of claim 1, wherein the reducing agent comprises a metal.

9. The method of claim 1, wherein the reducing agent comprises a metal halide.

10. The method of claim 1, wherein the reducing agent comprises SnCl₂ or a hydrated SnCl₂.

11. The method of claim 1, wherein the substantially insoluble crosslinked resin comprises a plurality of exchange moieties.

12. The method of claim 1, wherein the substantially insoluble crosslinked resin comprises a plurality of exchange moieties, each comprising one or more carboxylate groups.

13. The method of claim 1, wherein the solvent comprises water.

14. The method of claim 1, wherein the conditions and time sufficient to produce the radio-labeled chelate comprise heating the mixture above about 75° C. for about 10 minutes or more.

15. The method of claim 1, wherein the combining and reacting are performed in a tubular structure having capacity of less than 3 mL.

16. The method of claim 1, wherein the combining and reacting are performed in a tubular structure.

17. The method of claim 1, wherein the separating is performed by eluting the mixture in a manner that the resin is excluded, and the resulting mixture is collected substantially free of the resin.

18. The method of claim 17, wherein the eluting is performed by spinning a tubular structure.

19. The method of claim 17, further comprising passing the resulting mixture through a second substantially insoluble crosslinked resin different from the first resin in a manner that undesired products are substantially separated from the desired radio-labeled chelate.

20. The method of claim 19, further comprising eluting the radio-labeled chelate from the second resin using an anhydrous solvent.

21. The method of claim 20, wherein the anhydrous solvent is dimethyl formamide or dimethyl sulfoxide.

22. The method of claim 20, further comprising converting the radio-labeled chelate having a first conjugatable group to a second radio-labeled chelate having a second conjugatable group more reactive than the first conjugatable group by reacting the conjugatable group with one or more reagents to produce a mixture comprising the second radio-labeled chelate.

23. The method of claim 22, wherein the one or more reagents comprise N,N,N′N′-tetramethyl-O-(N-succinimidyl)uronium tetrafluoroborate, and diisopropylethylamine, and wherein the more reactive conjugatable group comprises an NHS ester.

24. The method claim 22, further comprising passing the mixture comprising the second radio-labeled chelate having the more reactive conjugatable group through one or more resins to remove undesired products and to provide the second radio-labeled chelate having the more reactive conjugatable group in a high purity form in a polar, substantially anhydrous solvent.

25. The method of claim 24, wherein the anhydrous solvent is DMF or DMSO.

26. The method of claim 24, having a total elapsed time of 25 minutes or less.

27. A method of making a radio-labeled material, the method comprising:
combining a chelating material having a conjugatable group or a protected conjugatable group with a radioactive metal-containing material, and a solvent to provide a mixture;
reacting the mixture under conditions and for a time sufficient to produce a reaction mixture comprising a radio-labeled chelate having the conjugatable group or the protected conjugatable group; and
separating the radio-labeled chelate from the reaction mixture by passing the reaction mixture through one or more substantially insoluble crosslinked resins.

28. The method of claim 27, wherein the chelating material has between 2 and about 8 binding sites, each binding site comprising a nitrogen, oxygen, or sulfur atom.

29. The method of claim 27, wherein the chelating material comprises DOTA-Ser.

30. The method of claim 27, wherein the conjugatable group is a carboxylic acid group.

31. The method of claim 27, wherein the radioactive metal-containing material comprises a metal selected from the group consisting of In, Y, Gd, Eu, a lanthanide, and mixtures thereof.

32. The method of claim 27, wherein the radioactive metal-containing material comprises ¹¹¹InCl₃.

33. The method of claim 27, wherein the mixture further includes a buffer.

34. The method of claim 33, wherein the buffer comprises ammonium acetate.

35. The method of claim 27, wherein the solvent comprises water.

36. The method of claim 27, wherein the conditions and time sufficient to produce the radio-labeled chelate comprise heating the mixture above about 75° C. for about 10 minutes or more.

37. The method of claim 27, wherein separating the radio-labeled chelate from the reaction mixture comprises passing the reaction mixture through a first resin configured to remove undesired metallic materials, and then passing through a second resin different from the first resin.

38. The method of claim 37, further comprising eluting the radio-labeled chelate from the second resin using an anhydrous solvent.

39. The method of claim 38, further comprising converting the radio-labeled chelate having a first conjugatable group to a second radio-labeled chelate having a second conjugatable group more reactive than the first conjugatable group by reacting the first conjugatable group with one or more reagents to produce a mixture comprising the second radio-labeled chelate.

40. The method claim 39, further comprising passing the mixture comprising the other radio-labeled chelate having the more reactive conjugatable group and other products through one or more resins to remove undesired products, and to provide the other radio-labeled chelate having the more reactive conjugatable group in a high purity form in a substantially anhydrous solvent.

41. A radio-labeled composition comprising a radio-labeled chelate having a conjugatable group or a protected conjugatable group dissolved in an anhydrous solvent, wherein a purity of the radio-labeled chelate is greater than 92.5 percent.

42. The radio-labeled composition of claim 41, wherein the conjugatable group is an NHS ester.

43. The radio-labeled composition of claim 42, wherein the purity of the radio-labeled chelate is 98.0 percent or greater.

44. The radio-labeled composition of claim 41, wherein the radio-labeled chelate having the conjugatable group or the protected conjugatable group comprises MAS₃(MO), MAS₃(MO)—NHS, ¹¹¹In-DOTA-Ser, or ¹¹¹In-DOTA-NHS.

45. A radio-labeled material comprising a compound of Structure I 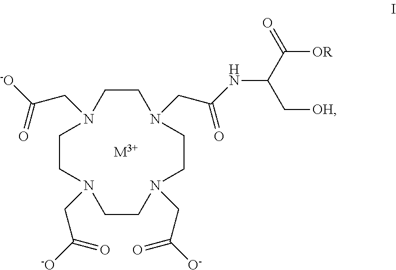
wherein
M is In, Y, Gd, Eu, or a lanthanide; and
R is H, a C1-C10 straight-chain or branched alkyl group, or N-succinimidyl.

46. The radio-labeled material of claim 45, wherein the compound has the structure: 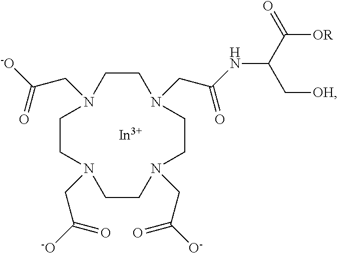
wherein
R is H or N-succinimidyl.

47. The radio-labeled material of claim 46, wherein the compound has the structure

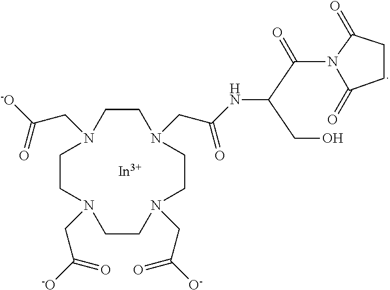

48. A reaction product of 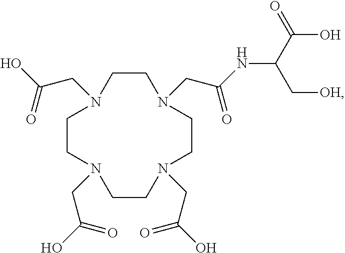
or an ester or salt thereof, and a metal compound comprising In, Y, Gd, Eu or a lanthanide.

49. The compound of claim 45, wherein the metal is radioactive.

50. A reaction product of the compound of claim 45 and a ligand.

51. A kit for preparing a radio-labeled material, comprising:
a chelating material;
a reducing agent; and
one or more cartridges, wherein the one or more cartridges comprise a substantially insoluble crosslinked resin.

52. The kit of claim 51, wherein the reducing agent and the chelating material are contained in a single vial, and wherein the reducing agent and the chelating agent are dissolved in a solvent.

53. The kit of claim 51, further comprising an anhydrous solvent.

54. The kit of claim 51, wherein the resin includes carboxylate groups.

55. The kit of claim 51, further comprising N,N,N′N′-tetramethyl-O-(N-succinimidyl)uronium tetrafluoroborate (TSTU), and diisopropylethylamine (DIEA).

56. The kit of claim 51, further comprising one or more ligands.

57. The compound of claim 48, wherein the metal is radioactive.

58. A reaction product of the compound of claim 48 and a ligand.
